(19) United States
(12) Patent Application Publication
(10) Pub. No.: US 2006/0142387 A1
(43) Pub. Date: Jun. 29, 2006

(54) Chemical compounds

(76) Inventors: Rodolfo Cadilla, Durham, NC (US); Andrew L. Larkin, Durham, NC (US); Eugene Lee Stewart, Durham, NC (US); Ryan Paul Trump, Durham, NC (US); Philip Stewart Turnbull, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/560,017

(22) PCT Filed: Jun. 9, 2004
(86) PCT No.: PCT/US04/18456

§ 371 Date: Dec. 8, 2005

Related U.S. Application Data
(60) Provisional application No. 60/477,256, filed on Jun. 10, 2003.

(51) Int. Cl. A61K 31/277; A61K 31/135; C07C 255/52

(52) U.S. Cl. 514/523; 514/657; 558/418; 564/428

(57) ABSTRACT

This invention relates to non-steroidal compounds that are modulators of androgen, glucocorticoid, mineralocorticoid, and progesterone receptors, and also to the methods for the making and use of such compounds.

### FIELD OF THE INVENTION

This invention relates to non-steroidal compounds that are modulators of androgen, glucocorticoid, mineralocorticoid, and progesterone receptors, and also to the methods for the making and use of such compounds.

### BACKGROUND OF THE INVENTION

Nuclear receptors are a class of structurally related gene expression modulators that act as ligand-dependent transcription factors (R. M. Evans, Science 240, 889 (1988)). The steroid receptors, namely the androgen receptor, the estrogen receptor, the glucocorticoid receptor, the mineralocorticoid receptor, and the progesterone receptor represent a subclass of the nuclear receptor superfamily. Nuclear receptor ligands in this subclass exert their effects by binding to an intracellular steroid hormone receptor. After the receptor-ligand complex is translocated to the nucleus of the cell, the complex binds to recognition sites on DNA, which allows for the modulation of certain genes.

Certain substances have demonstrated the ability to exhibit their activity in a tissue selective manner. In other words, tissue selectivity allows a nuclear receptor ligand to function as an agonist in some tissues, while having no effect or even an antagonist effect in other tissues. The term “selective receptor modulator” (SRM) has been given to these molecules. A synthetic compound that binds to an intracellular receptor and mimics the effects of the native hormone is referred to as an agonist. A compound that inhibits the effect of the native hormone is called an antagonist. The term “modulators” refers to compounds that have a spectrum of activities ranging from full agonism to partial agonism to full antagonism. The molecular basis for this tissue selective activity is not completely understood. Without being limited to any particular explanation, particular ligands put nuclear receptors in different conformational states. These states dictate the ability of coactivators, corepressors, and other proteins to be recruited by the nuclear receptor (“NR”). The unique cofactor-NR ensembles are the gene transcription factors that are thought to modulate tissue selective effects.

Ligand-mediated effects through the action of nuclear receptors are not limited to the classical genotropic mechanism outlined above. It is thought that some, if not all, of the separation of anabolic and general homeostatic effects from the stimulation of sexual tissues can be explained by a particular ligand's ability to potentiate non-genotropic pathways. One example of liganded nuclear receptor induction of non-genotropic pathways is found in the work of S. C. Manolagas et al., Cell, 104, 719-730. The action of a sex steroid NR on osteoblasts and other cell types is shown to involve the Src/Shc/ERK signaling pathway. This activity is mediated through the ligand binding domain of the sex steroid nuclear receptor alone. The NR DNA-binding domain is not required to attenuate etoposide-induced apoptosis in HeLa cells. An NR lacking the DNA binding domain cannot function in the classical mode, acting as a transcription factor.

Nuclear receptor steroid ligands are known to play important roles in the health of both men and women. In regard to men's health, testosterone (T) and dihydrotestosterone (DHT), for example, are endogenous steroidal ligands for the androgen receptor that likely play a role in every tissue type found in the mammalian body. During the development of the fetus, androgens play a role in sexual differentiation and development of male sexual organs. Further sexual development is mediated by androgens during puberty. Androgens play diverse roles in the adult including stimulation and maintenance of male sexual accessory organs and maintenance of the musculoskeletal system. Cognitive function, sexuality, aggression, and mood are some of the behavioral aspects mediated by androgens. Androgens affect the skin, bone, and skeletal muscle, as well as blood lipids and blood cells.

The study of androgen action and male reproductive dysfunction continues to expand significantly. In fact, only recently has the definition of a disease state been associated with hormonal changes that occur in aging men. This syndrome, previously referred to as Andropause, has more recently been described as Androgen Deficiency in the Aging Male, or “ADAM” (A. Morales and J. L. Tenover, Urologic Clinics of North America (November 2002) 29(4) 975.) The onset of ADAM is unpredictable and its manifestations are subtle and variable. Clinical manifestations of ADAM include fatigue, depression, decreased libido, erectile dysfunction as well as changes in cognition and mood.

Published information indicates that androgen replacement therapy (ART) in men may have benefits in terms of improving body composition parameters (e.g. bone mineral density, increasing muscle mass, and strength) as well as improving libido and mood in some men. Therefore, andrologists and other specialists are increasingly using ART for the treatment of the symptoms of ADAM—though there is due caution given androgen's, like testosterone, potential side effects. Nonetheless, there is increasing scientific rational of and evidence for androgen deficiency and treatment in the aging male. Current testosterone-based ART therapies include injections, skin patches, gel-based formulations, and oral preparations. All of these therapies are somewhat efficacious in the treatment of ADAM, but, due to the dramatic fluctuations in plasma T-levels following treatment, success with these therapies has been variable.

Testosterone replacement products, such as AndroGel® (1% testosterone gel CIII, marketed by Solvay Pharmaceuticals) are emerging as a treatment of choice among physicians. Such products, however, fail to correctly mimic physiological testosterone levels and have potential side effects including exacerbation of pre-existing sleep apnoea, polycythemia, and/or gynaecomastia. Furthermore, the longer-term side effects on target organs such as the prostate or the cardiovascular system are yet to be fully elucidated. Importantly, the potential carcinogenic effects of testosterone on the prostate prevent many physicians from prescribing it to older men (i.e. age >60 years) who, ironically, stand to benefit most from treatment. Also, all of the existing treatment options have fundamental problems with their delivery mechanism. The need for a novel selective androgen receptor modulator (SARM) is obviated by the potential side effect profile manifested in conventional treatments. A SARM would ideally have all the beneficial effects of endogenous androgens, while sparing sexual accessory organs, specifically the prostate.

In regard to female health, progesterone, the endogenous ligand for the progesterone receptor (“PR”), plays an important role in female reproduction during the various stages of the ovarian cycle and during pregnancy. Among other things, progesterone prepares the endometrium for implantation, regulates the implantation process, and helps maintain pregnancy. The therapeutic use of synthetic versions of progesterone (progestins) stems from progesterone's ability to regulate endometrial proliferation. In fact, progestins are included as part of hormone replacement therapy (“HRT”) in women to reduce the incidence of endometriosis. Unfortunately, the effectiveness of therapy is tempered by undesired side-effect profiles. Chronic progestin therapy or continuous estrogen replacement regimens are often associated with increased bleeding. Excessive stimulatory effects on the endometrial vasculature may result in proliferation and fragility.

Compounds that modulate the effects of progesterone binding to PR are believed useful in the treatment and/or prophylaxis of endometriosis and uterine fibroid processes. Progesterone receptor antagonists such as mifepristone, also known as RU-486, and other PR modulators can inhibit endometrial proliferation at high estradiol concentrations in primates. Human clinical data with mifepristone supports the efficacy of a PR antagonist in endometriosis (D. R. Grow et. al., J. Clin. Endoctin. Metab. 1996, 81). Despite enthusiasm for its use, RU-486 also acts as a potent ligand for the glucocorticoid receptor (“GR”). This cross-reactivity with the GR is associated with homeostatic imbalances.

Thus, modulators of nuclear steroid hormones that are highly specific for one receptor could offer greater benefit with less side effects in the treatment of both female and male related hormone responsive diseases.

### SUMMARY OF INVENTION

The present invention includes compounds of formula (I): 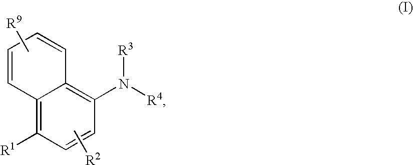
including salts, solvates, and physiologically functional derivatives thereof, wherein
R¹ is cyano, nitro, halogen, haloalkyl, heterocyclyl, hydroxy, alkoxy, haloalkoxy, —OC(O)R⁶, —CO₂R⁶, —CONHR⁶, —C(O)R⁶, —S(O),R⁶, —SO₂N(R⁶)₂, —NHC(O)R⁶, or —NHSO₂R⁶;
R² is H, cyano, nitro, halogen, haloalkyl, alkyl, alkenyl, alkynyl, hydroxy, alkoxy, haloalkoxy, —OC(O)R⁶, or aryl;
R³ and R⁴each are independently —(CH₂)ₓ—R⁵,
where x is 0 to 6, and
R⁵ is selected from H, alkyl, hydroxy, haloalkyl, alkenyl, alkynyl, cycloalkyl, heterocyclyl, —C(O)OR⁷, or —N(R⁸)₂;
each R⁶ independently is H, alkyl, alkenyl, alkynyl, cycloalkyl, heterocyclyl, aryl, aralkyl, heteroaryl, or heteroaralkyl;
each R⁷ independently is H, alkyl, cycloalkyl, aryl;
each R⁸ independently is H or alkyl; and
R⁹ is H, cyano, nitro, halogen, haloalkyl, alkyl, alkenyl, alkynyl, hydroxy, alkoxy, haloalkoxy, —OC(O)R⁸, or aryl.

Preferably, R¹ is cyano, nitro, or halogen. In one embodiment, R³ is —(CH₂)ₓ—R⁵, x is 0, and R⁵ is H, alkyl, or haloalkyl. Haloalkyl, preferably, is trifluoromethyl or trifluoroethyl. In one embodiment R³ is —(CH₂)ₓ—R⁵, x is 1, and R⁵ is cycloalkyl. In one embodiment R⁴ is —(CH₂)ₓ—R⁵, x is 0, and R⁵ is alkyl, cycloalkyl, or hydroxy. In one embodiment R⁴ is —(CH₂)ₓ—R⁵, x is 1 to 6, and R⁵ is alkyl, alkenyl, haloalkyl, hydroxy, cycloalkyl, heterocyclyl, heteroaryl, or —N(R¹⁰)₂ where each R¹⁰ is a C₁-C₆ alkyl group.

Preferred compounds of the present invention include:
N-(cyclopropylmethyl)-N-(4-nitro-1-naphthyl)-N-propylamine;
N-cyclohexyl-N-methyl-4-nitro-1-naphthalenamine;
N-(4-nitro-1-naphthyly-N,N-dipropylamine;
N-butyl-N-methyl-N-(4-nitro-1-naphthyl)amine;
4-[ethyl(2-methyl-2-propenyl)amino]-1-naphthonitrile;
N-butyl-N-ethyl-4-nitro-1-naphthalenamine;
4-[butyl(methyl)amino]-1-naphthonitrile;
4-[(cyclopropylmethyl)(propyl)amino]-1-naphthonitrile;
N¹-ethyl-N²,N²-dimethyl-N¹-(4-nitro-naphthyl)-1,2-ethanediamine;
4-(propylamino)-1-naphthonitrile;
4-[(3-hydroxypropyl)amino]-1-naphthonitrile;
3-[(4-nitro-1-naphthyl)amino]propan-1-ol;
4-[(cyclopropylmethyl)amino]-1-naphthalenecarbonitrile;
4{(cyclopropylmethyl)[3-(1-piperidinyl)propyl]amino}1-naphthalenecarbonitrile trifluoroacetate;
4-[(cyclopropylmethyl)(3-hydroxypropyl)amino]-1-naphthalenecarbonitrile;
4-nitro-N-(2,2,2-trifluoroethyl)-1-naphthalenamine;
4-bromo-N-(2,2,2-trifluoroethyl)-1-naphthalenamine;
4-bromo-N,N-bis(2,2,2-trifluoroethyl)-1-naphthalenamine;
4-[(2,2,2-trifluoroethyl)amino]-1-naphthalenecarbonitrile;
4-[bis(2,2,2-trifluoroethyl)amino]-1-naphthalenecarbonitrile;
4-[propyl(2,2,2-trifluoroethyl)amino]-1-naphthalenecarbonitrile;
4-[2-propen-1-yl(2,2,2-trifluoroethyl)amino]-1-naphthalenecarbonitrile; and
4-((2-hydroxyethyl)(2,2,2-trifluoroethyl)amino]-1-naphthalenecarbonitrile.

Another aspect of the present invention includes a compound substantially as hereinbefore defined with reference to any one of the Examples.

Another aspect of the present invention includes a pharmaceutical composition comprising a compound of the present invention and a pharmaceutically acceptable carrier.

Another aspect of the present invention includes a compound of the present invention for use as an active therapeutic substance.

Another aspect of the present invention includes a compound of the present invention for use in the treatment or prophylaxis of conditions or disorders that respond to selective androgen receptor modulation.

Another aspect of the present invention includes a compound of the present invention for use in the treatment or prophylaxis of osteoporosis, muscle wasting, frailty, cardiovascular disease, breast cancer, uterine cancer, prostate hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM.

Another aspect of the present invention includes the use of a compound of the present invention in the manufacture of a medicament for use in the treatment or, prophylaxis of conditions or disorders that respond to selective androgen receptor modulation.

Another aspect of the present invention includes using a compound according to the present invention in the manufacture of a medicament for use in the treatment or prophylaxis of osteoporosis, muscle wasting, frailty, cardiovascular disease, breast cancer, uterine cancer, prostatic hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM.

Another aspect of the present invention includes a method for the treatment or prophylaxis of conditions or disorders that respond to selective androgen receptor modulation comprising the administration of a compound according to the present invention.

Another aspect of the present invention includes a method for the treatment or prophylaxis of osteoporosis, muscle wasting, frailty, cardiovascular disease, breast cancer, uterine cancer, prostatic hyperplasia, prostate cancer, dyslipidemia, menopausal vasomotor conditions, urinary incontinence, artherosclerosis, libido enhancement, depression, uterine fibroid disease, aortic smooth muscle cell proliferation, endometiosis, or ADAM comprising the administration of a compound according to the present invention.

The compounds of the present invention modulate the function of the nuclear hormone receptors, particularly the androgen receptor (“AR”). The present invention includes compounds that are selective agonists, partial agonists, antagonists, or partial antagonists of the AR. Compounds of the present invention are useful in the treatment of AR-associated diseases and conditions, for example, a disease or condition that is prevented, alleviated, or cured through the modulation of the function or activity of AR. Such modulation may be isolated within certain tissues or widespread throughout the body of the subject being treated.

An aspect of the present invention is the use of the compounds of the present invention for the treatment or prophylaxis of a variety of disorders including, but not limited to, osteoporosis and/or the prevention of reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), dry eye, sarcopenia, chronic fatigue syndrome, chronic myaligia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, menopausal vasomotor conditions, urinary incontinence, artherosclerosis, libido enhancement, sexual dysfunction, depression, nervousness, irritability, stress, reduced mental energy and low self-esteem, improvement of cognitive function, endometriosis, polycystic ovary syndrome, counteracting preeclampsia, premenstral syndrome, contraception, uterine fibroid disease, aortic smooth muscle cell proliferation, male hormone replacement, or ADAM.

### DETAILED DESCRIPTION OF THE PREFERRED EMBODIMENT

Terms are used within their accepted meanings. The following definitions are meant to clarify, but not limit, the terms defined.

As used herein the term “alkyl” refers to a straight or branched chain hydrocarbon, preferably having from one to twelve carbon atoms, which may be optionally substituted, with multiple degrees of substitution included within the present invention. Examples of “alkyl” as used herein include, but are not limited to, methyl, ethyl, propyl, isopropyl, isobutyl, n-butyl, tert-butyl, isopentyl, n-pentyl, and substituted versions thereof.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain is aliphatic hydrocarbon containing one or more carbon-to-carbon double bonds that may be optionally substituted, with multiple degrees of substitution included within the present invention. Examples include, but are not limited to, vinyl and the like and substituted versions thereof.

As used herein the term “alkynyl” refers to a straight or branched chain aliphatic hydrocarbon containing one or more carbon-to-carbon triple bonds that may be optionally substituted, with multiple degrees of substitution included within the present invention. Examples include, but are not limited to, ethynyl and the like and substituted versions thereof.

As used herein, the term “alkylene” refers to a straight or branched chain divalent hydrocarbon radical, preferably having from one to ten carbon atoms. Alkylene groups as defined herein may optionally be substituted, with multiple degrees of substitution included within the present invention. Examples of “alkylene” as used herein include, but are not limited to, methylene (—CH₂—), ethylene (—CH₂—CH₂—), propylene (—CH₂—CH₂—CH₂—) as well as substituted and/or branched versions.

As used herein, the term “cycloalkyl” refers to an optionally substituted non-aromatic cyclic hydrocarbon ring, which optionally includes an alkylene linker through which the cycloalkyl may be attached, with multiple degrees of substitution included within the present invention. Exemplary “cycloalkyl” groups include, but are not limited to, cyclopropyl, cyclobutyl, cyclopentyl, cyclohexyl, cycloheptyl, and substituted versions thereof.

As used herein, the terms “heterocycle,” “heterocyclic” or “heterocyclyl” refers to a mono- or poly-cyclic ring system containing optionally one or more degrees of unsaturation, but not to overlap with heteroaryl, and also containing optionally one or more heteroatoms. Preferred heteroatoms include N, O, and/or S, including N-oxides, sulfur oxides, and dioxides. Preferably the ring is three to ten-membered and is either saturated or has one or more degrees of unsaturation. Optionally, as used herein, the heterocycle may be substituted, with multiple degrees of substitution being allowed. Such rings may be optionally fused to one or more of another “heterocyclic” ring(s), heteroaryl ring(s), aryl ring(s), or cycloalkyl ring(s). Examples of “heterocyclic” groups include, but are not limited to, tetrahydrofuran, pyran, 1,4-dioxane, 1,3-dioxane, piperidine, pyrrolidine, morpholine, tetrahydrothiopyran, and tetrahydrothiophene.

As used herein, the term “aryl” refers to an optionally substituted benzene ring or to an optionally substituted fused benzene ring system, for example anthracene, phenanthrene, or naphthalene ring systems. Multiple degrees of substitution are included within the present definition. Examples of “aryl” groups include, but are not limited to, phenyl, 2-naphthyl, 1-naphthyl, biphenyl, and substituted derivatives thereof. The term “aralkyl” refers to an aryl group as defined herein attached through an alkylene linker, such as, for example, benzyl.

As used herein, the term “heteroaryl” refers to an optionally substituted monocyclic five to seven membered aromatic ring, or to an optionally substituted fused bicyclic aromatic ring system comprising two of such aromatic rings, which contain one or more nitrogen, sulfur, and/or oxygen atoms, where N-oxides, sulfur oxides, and dioxides are permissible heteroatom substitutions. Multiple degrees of substitution are included within the present definition. Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and substituted versions thereof. The term “heteroaralkyl” refers to a heteroaryl group as defined herein attached through an alkylene linker.

As used herein the term “halogen” refers to fluorine, chlorine, bromine, or iodine.

As used herein the term “haloalkyl” refers to an alkyl group, as defined herein that is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents such as —CF₃, —CH₂—CH₂—F, and the like.

As used herein the term “hydroxy” refers to a group —OH.

As used herein the term “alkoxy” refers to a group —ORₐ, where Rₐ is alkyl as defined.

As used herein the term “haloalkoxy” refers to a group —ORₐ, where Rₐ is haloalkyl as defined.

As used herein the term “aryloxy” refers to a group —R_{b}, where R_{b} is aryl as defined.

As used herein the term “heteroaryloxy” refers to a group —OR_{b}, where R_{b} is heteroaryl as defined.

As used herein the term “nitro” refers to the group —NO₂.

As used herein the term “cyano” refers to the group —CN.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted. Rather, those of ordinary skill in the art will appreciate that the phrase is included to provide for obvious modifications, which are encompassed within the scope of the appended claims.

Exemplary optional substituents include acyl; alkyl; alkenyl; alkynyl; alkylsulfonyl; alkoxy; cyano; halogen; haloalkyl; hydroxy; nitro; aryl, which may be further substituted with acyl, alkoxy, alkyl, alkenyl, alkynyl, alkylsulfonyl, cyano, halogen, haloalkyl, hydroxy, or nitro; heteroaryl, which may be further substituted with acyl, alkoxy, alkyl, alkenyl, alkynyl, alkylsulfonyl, cyano, halogen, haloalkyl, hydroxy, or nitro; arylsulfonyl, which may be further substituted with acyl, alkoxy, alkyl, alkenyl, alkynyl, alkylsulfonyl, cyano, halogen, haloalkyl, hydroxy, or nitro; heteroarylsulfonyl, which may be further substituted with acyl, alkoxy, alkyl, alkenyl, alkynyl, alkylsulfonyl, cyano, halogen, haloalkyl, hydroxy, or nitro; aryloxy, which may be further substituted with acyl, alkoxy, alkyl, alkenyl, alkynyl, alkylsulfonyl, cyano, halogen, haloalkyl, hydroxy, or nitro; heteroaryloxy, which may be further substituted with acyl, alkoxy, alkyl, alkenyl, alkynyl, alkylsulfonyl, cyano, halogen, haloalkyl, hydroxy, or nitro; or —N(R*)₂; where for each occurrence R* is independently selected from H, alkyl, alkenyl, alkynyl, cycloalkyl, heterocyclyl, aryl, aralkyl, heteroaryl, heteroaralkyl, alkylsulfonyl, arylsulfonyl, or heteroarylsulfonyl, where each occurrence of such aryl or heteroaryl may be substituted with one or more acyl, alkoxy, alkyl, alkenyl, alkylsulfonyl, cyano, halogen, haloalkyl, hydroxy, or nitro, or the two R's may combine to form a ring, optionally having additional heteroatoms, optionally having one or more degrees of unsaturation, and optionally being further substituted with acyl, alkoxy, alkyl, alkenyl, alkynyl, alkylsulfonyl, cyano, halogen, haloalkyl, hydroxy, or nitro.

The compounds of formulas (I) may crystallize in more than one form, a characteristic known as polymorphism, and such polymorphic forms (“polymorphs”) are within the scope of formula (I). Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by formula (I), as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts-include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The biological or medical response may be considered a prophylactic response or a treatment response. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of formula (I), as well as salts, solvates, and physiological functional derivatives thereof, may be administered as the raw chemical. Additionally, the active ingredient may be presented as a pharmaceutical composition.

Accordingly, the invention further provides pharmaceutical compositions that include effective amounts of compounds of the formula (I) and salts, solvates, and physiological functional derivatives thereof, and one or more pharmaceutically acceptable carriers, diluents, or excipients. The compounds of formula (I) and salts, solvates, and physiologically functional derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the formula (I) or salts, solvates, and physiological functional derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of formula (I) for the treatment of humans suffering from frailty, generally, should be in the range of 0.1 to 100 mg/kg body weight of recipient (mammal) per day. More usually the effective amount should be in the range of 1 to 10 mg/kg body weight per day. Thus, for a 70 kg adult mammal the actual amount per day would usually be from 70 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of formula (I) per se. Similar dosages should be appropriate for treatment or prophylaxis of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of w,ax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) and salts, solvates, and physiological functional derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of formula (I) and salts, solvates, and physiologically functional derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, and physiologically functional derivatives thereof, may be employed alone or in combination with other therapeutic agents for the treatment of the above-mentioned conditions. For example, in frailty therapy, combination may be had with other anabolic or osteoporosis therapeutic agents. As one example, osteoporosis combination therapies according to the present invention would thus comprise the administration of at least one compound of formula (I) or a salt, solvate, or physiologically functional derivative thereof, and the use of at least one other osteoporosis therapy. As a further example, combination therapies according to the present invention include the administration of at least one compound of formula (I) or a salt, solvate, or physiologically functional derivative thereof, and at least one other osteoporosis treatment agent, for example, an anti-bone resorption agent. The compound(s) of formula (I) and the other pharmaceutically active agent(s) may be administered together or separately and, when administered separately, administration may occur simultaneously or sequentially, in any order. The amounts of the compound(s) of formula (I) and the other pharmaceutically active agent(s) and the relative timings of administration will be selected in order to achieve the desired combined therapeutic effect. The administration in combination of a compound of formula (I) salts, solvates, or physiologically functional derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

Another potential osteoporosis treatment agent is a bone building (anabolic) agent. Bone building agents can lead to increases in parameters such as bone mineral density that are greater than those than can be achieved with anti-resorptive agents. In some cases, such anabolic agents can increase trabecular connectivity leading to greater structural integrity of the bone.

Other potential therapeutic combinations include the compounds of the present invention combined with other compounds of the present invention, growth promoting agents, growth hormone secretagogues, growth hormone releasing factor and its analogs, growth hormone and its analogs, somatomedins, alpha-ardenergic agonists, serotonin 5-HTD agonists, agents that inhibit somatostatin or its release, 5-α-reductase inhibitors, aromatase inhibitors, GnRH agonists or antagonists, parathyroid hormone, bisphosphonates, estrogen, testosterone, SERMs, progesterone receptor agonists or antagonists, and/or with other modulators of nuclear hormone receptors.

One skilled in the art will acknowledge that although the compounds embodied herein will be used as selective agonists, partial agonists, and antagonists, compounds with mixed steroid activities may also be employed.

The compounds of the present invention may be used in the treatment of a variety of disorders and conditions and, as such, the compounds of the present invention may be used in combination with a variety of other suitable therapeutic agents useful in the treatment or prophylaxis of those disorders or conditions. Non-limiting examples include combinations of the present invention with anti-diabetic agents, anti-osteoporosis agents, anti-obesity agents, anti-inflammatory agents, anti-anxiety agents, anti-depressants, anti-hypertensive agents, anti-platelet agents, anti-thrombotic and thrombolytic agents, cardiac glycosides, cholesterol or lipid lowering agents, mineralocorticoid receptor antagonists, phosphodiesterase inhibitors, kinase inhibitors, thyroid mimetics, anabolic agents, viral therapies, cognitive disorder therapies, sleeping disorder therapies, sexual dysfunction therapies, contraceptives, cytotoxic agents, radiation therapy, anti-proliferative agents, and anti-tumor agents. Additionally, the compounds of the present invention may be combined with nutritional supplements such as amino acids, triglycerides, vitamins, minerals, creatine, piloic acid, carnitine, or coenzyme Q10.

An aspect of the present invention is the use of the compounds of the present invention for the treatment or prophylaxis of a variety of disorders including, but not limited to, osteoporosis and/or the prevention of reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), dry eye, sarcopenia, chronic fatigue syndrome, chronic myaligia, acute fatigue syndrome, acceleration of wound healing, maintenance of sensory function, chronic liver disease, AIDS, weightlessness, bum and trauma recovery, thrombocytopenia, short bowel syndrome, irritable bowel syndrome, inflammatory bowel disease, Crohn's disease and ulcerabve colitis, obesity, eating disorders including anorexia associated with cachexia or aging, hypercortisolism and Cushing's syndrome, cardiovascular disease or cardiac dysfunction, congestive heart failure, high blood pressure, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, menopausal vasomotor conditions, urinary incontinence, artherosclerosis, libido enhancement, sexual dysfunction, depression, nervousness, irritability, stress, reduced mental energy and low self-esteem, improvement of cognitive function, endometriosis, polycystic ovary syndrome, counteracting preeclampsia, premenstral syndrome, contraception, uterine fibroid disease, aortic smooth muscle cell proliferation, male hormone replacement, or ADAM.

In particular, the compounds of the present invention are believed useful, either alone or in combination with other agents, in the treatment of and use as male and female hormone replacement therapy, hypogonadism, osteoporosis, muscle wasting, wasting diseases, cancer cachexia, frailty, prostatic hyperplasia, prostate cancer, breast cancer, menopausal and andropausal vasomotor conditions, urinary incontinence, sexual dysfunction, erectile dysfunction, depression, uterine fibroid disease, and/or endometriosis, treatment of acne, hirsutism, stimulation of hematopoiesis, male contraception, impotence, and as anabolic agents.

Another aspect of the present invention thus also provides compounds of formula (I) and salts, solvates, or physiologically functional derivatives thereof, for use in medical therapy. Particularly, the present invention provides for the treatment or prophylaxis of disorders mediated by androgenic activity. More particularly, the present invention provides through the treatment or prophylaxis of disorders responsive to tissue-selective anabolic and or androgenic activity. A further aspect of the invention provides a method of treatment or prophylaxis of a mammal suffering from a disorder mediated by androgenic activity, which includes administering to said subject an effective amount of a compound of formula (I) or a salt, solvate, or physiologically functional derivative thereof.

A further aspect of the invention provides a method of treatment or prophylaxis of a mammal requiring the treatment or prophylaxis of a variety of disorders including, but not limited to, osteoporosis and/or the prevention of reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), dry eye, sarcopenia, chronic fatigue syndrome, chronic myaligia, acute fatigue syndrome, acceleration of wound healing, maintenance of sensory function, chronic liver disease, AIDS, weightlessness, bum and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, menopausal vasomotor conditions, urinary incontinence, artherosclerosis, libido enhancement, sexual dysfunction, depression, nervousness, irritability, stress, reduced mental energy and low self-esteem, improvement of cognitive function, endometriosis, polycystic ovary syndrome, counteracting preeclampsia, premenstral syndrome, contraception, uterine fibroid disease, aortic smooth muscle cell proliferation, male hormone replacement, or ADAM. Preferably the compounds of the present invention are used as male and female hormone replacement therapy or for the treatment or prevention of hypogonadism, osteoporosis, muscle wasting, wasting diseases, cancer cachexia, frailty, prostatic hyperplasia, prostate cancer, breast cancer, menopausal and andropausal vasomotor conditions, urinary incontinence, sexual dysfunction, erectile dysfunction, depression, uterine fibroid disease, and/or endometriosis, treatment of acne, hirsutism, stimulation of hematopoiesis, male contraception, impotence, and as anabolic agents, which use includes administering to a subject an effective amount of a compound of formula (I) or a salt, solvate, or physiologically functional derivative thereof. The mammal requiring treatment with a compound of the present invention is typically a human being.

The compounds of this invention may be made by a variety of methods, including well-known standard synthetic methods. Illustrative general synthetic methods are set out below and then specific compounds of the invention are prepared in the working Examples.

In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of formula (I).

Those skilled in the art will recognize if a stereocenter exists in compounds of formula (I). Accordingly, the present invention includes all possible stereoisomers and includes not only racemic compounds but the individual enantiomers as well. When a compound is desired as a single enantiomer, such may be obtained by stereospecific synthesis or by resolution of the final product or any convenient intermediate. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

Representative AR modulator compounds, agonists, partial agonists, and antagonists according to the current invention include:
N-(cyclopropylmethyl)-N-(4-nitro-1-naphthyl)-N-propylamine;
N-cyclohexyl-N-methyl-4-nitro-1-naphthalenamine;
N-(4-nitro-1-naphthyl)-N,N-dipropylamine;
N-butyl-N-methyl-N-(4-nitro-1-naphthyl)amine;
4-[ethyl(2-methyl-2-propenyl)amino]-1-naphthonitrile;
N-butyl-N-ethyl-4-nitro-1-naphthalenamine;
4-[butyl(methyl)amino]-1-naphthonitrile;
4-[(cyclopropylmethyl)(propyl)amino]-1-naphthonitrile;
N¹-ethyl-N²,N²-dimethyl-N¹-(4nitro-1-naphthyl)-1,2-ethanediamine;
4-(propylamino)-1-naphthonitrile;
4-[(3-hydroxypropyl)amino]-1-naphthonitrile;
3-[(4-nitro-1-naphthyl)amino]propan-1-ol;
4-[(cyclopropylmethyl)amino]-1-naphthalenecarbonitrile;
4-(cyclopropylmethyl)[3-(1-piperidinyl)propyl]amino-1-naphthalenecarbonitrile trifluoroacetate;
4-[(cyclopropylmethyl)(3-hydroxypropyl)amino]-1-naphthalenecarbonitrile;
4nitro-N-(2,2,2-trifluoroethyl)-1-naphthalenamine;
4-bromo-N-(2,2,2-trifluoroethyl)-1-naphthalenamine;
4-bromo-N,N-bis(2,2,2-trifluoroethyl)-1-naphthalenamine;
4-[(2,2,2-trifluoroethyl)amino]-1-naphthalenecarbonitrile;
4-[bis(2,2,2-trifluoroethyl)amino]-1-naphthalenecarbonitrile;
4-[propyl(2,2,2-trifluoroethyl)amino]-1-naphthalenecarbonitrile;
4-[2-propen-1-yl(2,2,2-trifluoroethyl)amino]-1-naphthalenecarbonitrile; and
4-[(2-hydroxyethyl)(2,2,2-trifluoroethyl)amino]-1-naphthalenecarbonitrile.

### ABBREVIATIONS

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:
g (grams);
L (liters);
μL (microliters);
M (molar);
Hz (Hertz);
mol (moles);
RT (room temperature);
min (minutes);
mp (melting point);
t_{R} (retention time);
TEA (triethylamine);
TFAA (trifluoroacetic anhydride);
CDCl₃ (deuterated chloroform);
SiO₂ (silica);
EtOAc (ethyl acetate);
HCl (hydrochloric acid);
mg (milligrams);
mL (milliliters);
psi (pounds per square inch);
mM (millimolar);
MHz (megahertz);
mmol (millimoles);
h (hours);
TLC (thin layer chromatography);
RP (reverse phase);
TFA (trifluoroacetic acid);
THF (tetrahydrofuran);
CD₃OD (deuterated methanol);
DMSO (dimethylsulfoxide);
atm (atmosphere);
CHCl₃ (chloroform);
Ac (acetyl);
DMF (N,N-dimethylformamide);
Cs₂CO₃ (cesium carbonate);
Et (ethyl);
MeOH (methanol);
PS (polymer supported);
satd (saturated).
Me (methyl);
EtOH (ethanol);
tBu (tert-butyl);
PPTS (pyridinium p-toluenesulfonate);
NMM (N-methyl morpholine);

Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad). 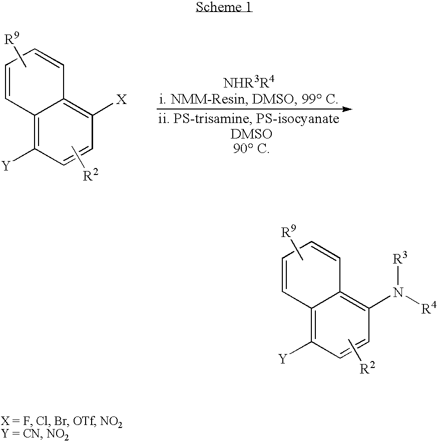
Method A

Compounds of formula (I) can be prepared starting from electron deficient arenes and utilizing solid supported reagents (Scheme 1). The requisite arenes are treated with primary or secondary non-cyclic amines in the presence of a solid supported base such as N-methyl morpholine to afford the corresponding aniline. Excess halo arene can be scavenged with polymer supported trisamine, while excess amine can be scavenged with polymer supported isocyanate. 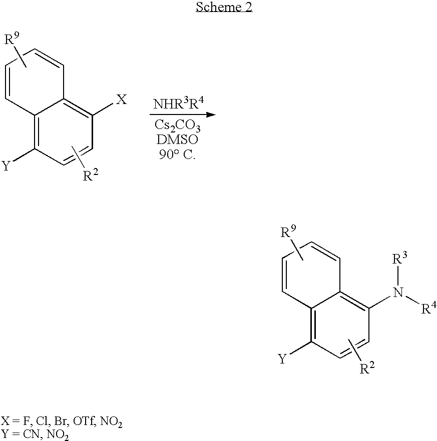
Method B

Compounds of formula (I) can also be prepared without the use of solid supported reagents (Scheme 2). The requisite electron deficient arenes are treated with primary or secondary non-cyclic amines in the presence of a base such as cesium carbonate to afford the corresponding aniline.

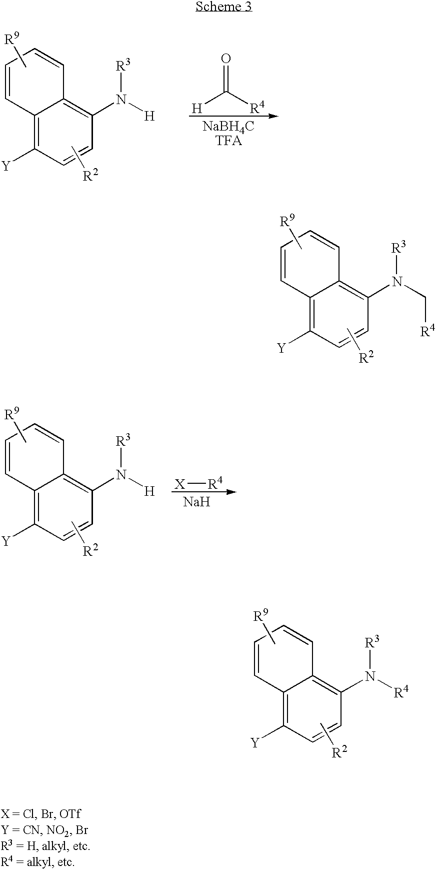

Compounds of formula (I) can be generated or further elaborated by reductive alkylation with an aldehyde and a reducing agent in the presence of an acid and/or by direct alkylation with an alkylating agent in the presence of base such as sodium hydride (Scheme 3).

### EXAMPLES

Method A Representative Procedure

### Example 1

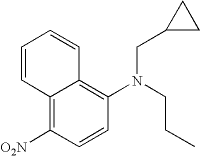

### N-(Cyclopropylmethyl)-N-(4-nitro-1-naphthyl)-N-propylamine

Solid supported N-methyl morpholine (PS-NMM) Resin (0.045 g, 180 μmol), a 2 M solution of (cyclopropylmethyl)propylamine in DMSO (120 μL, 0.24 mmol), and a 1 M solution of 4-chloro-1-nitronaphthalene in DMSO (150 μL, 0.12 mmol) were added to a well of a Robbins FlexChem square well plate. The plate was rotated for 20 h at 99° C. and cooled. To the well was added 1.2 mL of DMSO, solid supported benzylisocyanate (PS-Isocyanate) (0.136 g, 150 μmol), and solid supported tris amine (PS-Trisamine) (0.061 g, 150 μmol). The plate was rotated at 99° C. for 12 h. The solvent in the well was collected via filtration. The resins were rinsed with 0.5 mL DMSO, and the organic solutions were combined and concentrated in vacuo at 60° C. to afford an analytically pure yellow solid (0.032 g, 95%): ¹H NMR (CDCl₃, 400 MHz) δ 8.75 (d, J=7.6 Hz, 1H), 8.31 (d, J=8.5 Hz, 2H), 7.68 (dd, J=8.1, 8.1 Hz, 1H), 7.55 (dd, J=7.9, 7.9 Hz, 1H), 7.09 (d, J=8.4 Hz, 1H), 3.42 (t, J=7.1 Hz, 2H), 3.20 (d, J=6.8 Hz, 2H), 1.58 (sex, J=7.3 Hz, 2H ), 0.96 (sept, J=4.9 Hz, 1H), 0.90 (t, J=7.5 Hz, 3H), 0.45 (q, J=5.3 Hz, 2H), 0.04 (q, J=4.9 Hz, 2H).

TABLE 1
All entries in the table below were made according to method A.

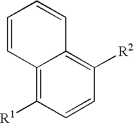

| | | | | MassSpec. |
|---|---|---|---|---|
| | | | | ES m/z |
| Example | R¹ | R² | Compound Name | (M + 1) |
| 2 | NO₂ | 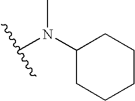 | N-cyclohexyl-N-methyl-4-nitro-1- naphthalenamine | 285 |
| 3 | NO₂ | 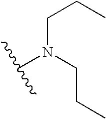 | N-(4-nitro-1-naphthyl)-N,N- dipropylamine | 273 |
| 4 | NO₂ | 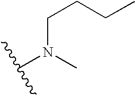 | N-butyl-N-methyl-N-(4-nitro-1- naphthyl)amine | 259 |
| 5 | CN | 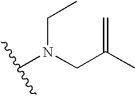 | 4-[ethyl(2-methyl-2- propenyl)amino]-1-naphthonitrile | 251 |
| 6 | NO₂ | 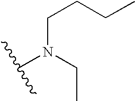 | N-butyl-N-ethyl-4-nitro-1- naphthalenamine | 273 |
| 7 | CN | 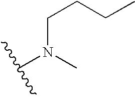 | 4-[butyl (methyl)amino]-1- naphthonitrile | 240 |

Method B Representative Procedure

### Example 1

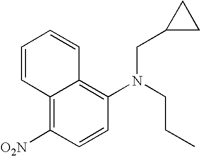 N-(Cyclopropylmethyl)-N-(4-nitro-1-naphthyl)-N-propylamine

A DMSO (0.5 mL) solution of 1-chloro-4-nitronaphthalene (0.054 g, 0.26 mmol, 1 equiv) was treated with cesium carbonate (0.120 g, 0.37 mmol, 1.4 equiv) and (cyclopropylmethyl)propylamine (0.035 g, 0.31 mmol, 1.2 equiv). After 3 h at 90° C., the cooled reaction was treated with H₂O (1 mL), and extracted with EtOAc (3×1 mL). Concentration was followed by radial chromatography (SiO₂, 1 mm plate, 90:10; Hex/EtOAc) to afford the title compound as a yellow solid (0.063 g, 95%) with analytical data that matched that for example 1 synthesized by method A.

### Example 8

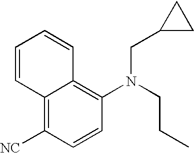

### 4-[(Cyclopropylmethyl)(propyl)amino]-1-naphthonitrile

Made in a manner similar to example 1, method B using 4-fluoro-1-naphthonitrile as the electron deficient arene component: ¹H NMR (CDCl₃, 400 MHz) δ 8.30 (d, J=6.8 Hz, 1H), 8.19 (d, J=8.2 Hz, 1H), 7.81 (d, J=8.1 Hz, 1H), 7.64 (dd, J=7.3, 7.3 Hz, 1H), 7.56 (dd, J=7.7, 7.7 Hz, 1H), 7.13 (d, J=7.9 Hz, 1H), 3.37 (t, J=7.1 Hz, 2H), 3.15 (d, J=6.4 Hz, 2H), 1.57 (sex, J=7.2 Hz, 2H), 0.96 (sept, J=5.2 Hz, 1H), 0.89 (t, J=7.3 Hz, 3H), 0.44-0.42 (m, 2H), 0.01-0.00 (m, 2H).

### Example 9

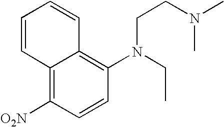

### N¹-Ethyl-N²,N²-dimethyl-N¹-(4-nitro-1-naphthyl)-1,2-ethanediamine

Made in a manner similar to example 1, method B using N¹-ethyl-N²,N²-dimethyl-1,2-ethanediamine as the amine component: ¹H NMR (CDCl₃, 400 MHz) δ 8.65 (d, J=8.8 Hz, 1H), 8.31 (d, J=8.4 Hz, 1H), 8.25 (d, J=7.9 Hz, 1H), 7.72 (dd, J=7.0, 7.0 Hz, 1H), 7.64 (dd, J=7.3, 7.3 Hz, 1H), 7.13 (d, J=8.4 Hz, 1H), 3.76 (t, J=6.4 Hz, 2H), 3.37 (q, J=7.1 Hz, 2H), 3.20 (t, J=3.2 Hz, 2H), 2.82 (s, 6H), 1.07 (t, J=7.1 Hz, 3H).

### Example 10

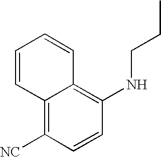

### 4-(Propylamino)-1-naphthonitrile

Synthesized in a manner similar to example 1, method B using 4-fluoro-1-naphthonitrile and propylamine: MS (ES) m/z 211 (M+1).

### Example 11

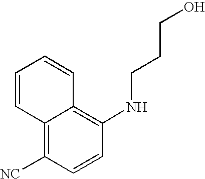

### 4-[(3-Hydroxypropyl)amino]-1-naphthonitrile

Prepared according to example 1, method B using 4-fluoro-1-naphthonitrile and 3-aminopropan-1-ol: MS (ES) m/z 225 (M−1).

### Example 12

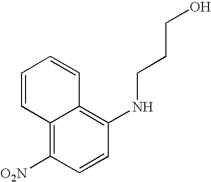

### 3-[(4-Nitro-1-naphthyl)amino]propan-1-ol

Prepared according to example 1, method B using 1-chloro-4-nitronaphthalene and 3-aminopropan-1-ol: MS (ES): m/z 245 (M−1).

### Example 13

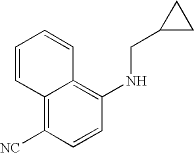

### 4-[(Cyclopropylmethyl)amino]-1-naphthalenecarbonitrile

Prepared according to example 1, method B using 4-fluoro-1-naphthonitrile and (cyclopropylmethyl)amine: MS (APCI) m/z 223 (M+1).

### Example 14

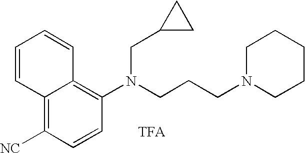

### 4-{(Cyclopropylmethyl)[3-(1-piperidinyl)propyl]amino}-1-naphthalenecarbonitrile trifluoroacetate

To a slurry of NaH (0.024 g of a 60% w/w suspension in mineral oil, 0.60 mmol) and NaI (0.030 g, 0.20 mmol) in dry DMF (1 mL) at room temperature was added example 13 (0.044 g, 0.20 mmol) in one portion. The mixture was stirred for 15 min and a solution of 1-(3-chloropropyl)piperidine (0.065 g, 0.40 mmol) in DMF (0.5 mL) was added. The mixture was heated at 60° C. for 12 h, cooled and poured into 10% v/v HCl. The whole was extracted with Et₂O (×2), treated with satd. Na₂CO₃ and extracted with Et₂O (×3). Ether extracts from the basic aqueous layer were combined and concentrated in vacuo. The residue was purified by preparative HPLC (C18 column, MeCN/water with 0.1% v/v TFA), affording 0.033 g of the title compound as an orange gum: MS (APCI) m/z 348 (M+1).

### Example 15

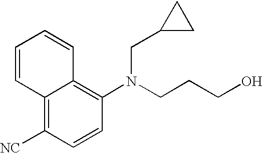

### 4-[(Cyclopropylmethyl)(3-hydroxypropyl)amino]-1-naphthalenecarbonitrile

To a slurry of NaH (0.018 g of a 60% w/w suspension in mineral oil, 0.45 mmol) in dry DMF (1 mL) at room temperature was added example 13 (0.067 g, 0.30 mmol) in one portion. The mixture was stirred 20 min and [(3-bromopropyl)oxy](1,1-dimethylethyl)dimethylsilane (0.080 mL, 0.36 mmol) was added via syringe. The mixture was stirred 20 h, poured into water and the whole was extracted with Et₂O (×3). The combined organic portions were washed (water, brine) and concentrated in vacuo. The residue was dissolved in EtOH (2 mL) and PPTS (0.030 g) was added. The mixture was heated at 80° C. in a sealed vial for 2 h, cooled, and concentrated in vacuo. The residue was purified by preparative HPLC (C18 column, MeCN/water with 0.1% v/v TFA), affording 0.037 g of the title compound as a colorless gum: MS (APCI) m/z 281 (M+1).

### Example 16

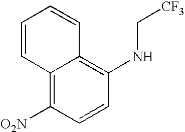

### 4-Nitro-N-(2,2,2-trifluoroethyl)-1-naphthalenamine

To a solution of 4-nitro-1-naphthalenamine (3.76 g, 20.0 mmol) in TFA (20 mL) at 0° C. was added NaBH₃CN (2.51 g, 40 mmol), portionwise over 10 min (CAUTION: exothermic reaction with hydrogen evolution). The mixture was stirred 5 min and trifluoroacetaldehyde hydrate (5.76 g, 40 mmol) was added, dropwise over 3 min. The flask was sealed with a rubber septum, and placed under balloon pressure of nitrogen. The cooling bath was removed and the mixture was stirred at room temp. for 12 h. The mixture was neutralized by slowly pouring into saturated NaHCO₃ (0° C.), and the whole was extracted with EtOAc (×3). The combined organic portions were washed (water, brine), dried over Na₂SO₄, filtered, and concentrated to dryness. Recrystallization from EtOAc/hexanes afforded 2.82 g of the title compound as bright yellow needles: MS (APCI) m/z 269 (M−1).

### Example 17

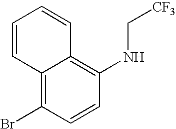

### 4-Bromo-N-(2,2,2-trifluoroethyl)-1-naphthalenamine

Synthesized as described in example 16 from 4-bromo-1-naphthalenamine: ¹H NMR (300 MHz, DMSO-d₈) δ 8.25 (d, J=8.7 Hz 1H), 7.42 (overlapping dd, 1H), 7.30-7.15 (m, 3H), 6.89 (d, J=7.9 Hz, 1H), 6.80 (t, J=6.6 Hz, 1H), 4.17 (overlapping qd, 2H).

### Example 18

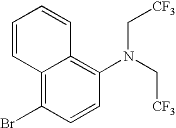

### 4-Bromo-N,N-bis(2,2,2-trifluoroethyl)-1-naphthalenamine

To a solution of example 17 (0.253 g, 0.832 mmol) in TFA (3 mL) at 0° C. was added NaBH₃CN (0.261 g, 4.16 mmol), portionwise over 3 min. The mixture was stirred 5 min and trifluoroacetaldehyde hydrate (0.214 g, 1.66 mmol) was added. The flask was sealed with a rubber septum, placed under balloon pressure of nitrogen and the cooling bath was removed. Excess trifluoroacetaldehyde hydrate (2.14 g, 16.6 mmol) was added to the stirred mixture via syringe pump over 15 h. The mixture was slowly poured into saturated NaHCO₃ (0° C.) and the whole was extracted with EtOAc (×3). The combined organic portions were washed (water, brine), dried over Na₂SO₄, filtered and concentrated in vacuo. The residue was purified by flash chromatography (EtOAc/hexanes), affording 0.267 g of the title compound as a white solid: ¹H NMR (300 MHz, acetone-d₆) δ 8.34 (d, J=8.5 Hz, 1H), 7.79 (d, J=7.6 Hz, 1H), 7.60 (overlapping ddd, 1H), 7.51 (d, J=7.6 Hz, 1H), 7.37 (overlapping ddd, 1H), 7.29 (d, J=8.3 Hz, 1H), 4.30 (q, J=9.1 Hz, 4H).

### Example 19

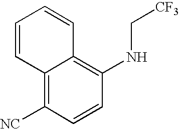

### 4-[(2,2,2-Trifluoroethyl)amino]-1-naphthalenecarbonitrile

Synthesized as described in example 16 from 4-amino-1-naphthalenecarbonitrile: ¹H NMR (300 MHz, CDCl₃) δ 8.22 (d, J=8.3 Hz, 1H), 7.87-7.78 (m, 2H), 7.70 (overlapping ddd, 1H), 7.61 (overlapping ddd, 1H), 6.71 (d, J=8.3 Hz, 1H), 5.19 (br. t, J=6.4 Hz, 1H), 4.05 (overlapping qd, 2H).

### Example 20

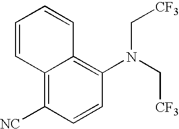

### 4-[Bis(2,2,2-trifluoroethyl)amino]-1-naphthalenecarbonitrile

Synthesized as described in example 18 using example 19: MS (EI) m/z 332 (M⁺, 86%), 263 ([M−CF₃]⁺, 100%).

### Example 21

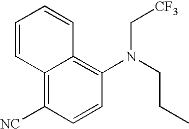

### 4-[Propyl(2,2,2-trifluoroethyl)amino]-1-naphthalenecarbonitrile

To a slurry of hexanes-washed NaH (0.033 g of a 60% w/w suspension in mineral oil; 0.82 mmol) in dry DMF (2 mL) at 0° C. was added a solution example 19 (0.102 g, 0.41 mmol) in DMF (1 mL), dropwise over 2 min. The mixture was stirred 20 min, 1-iodopropane (0.080 mL, 0.82 mmol) was added and the cooling bath was removed.

After stirring 12 h at rt, the mixture was poured into water and the whole extracted with ether (×3). The combined organic portions were washed (water, brine), dried over Na₂SO₄, filtered and concentrated in vacuo. The residue was purified by flash chromatography (EtOAc/hexanes), affording 0.076 g of the title compound as a colorless gum: ¹H NMR (400 MHz, MeOHd₄) δ 8.31 (d, J=8.3 Hz, 1H), 8.15 (d, J=8.3 Hz, 1H), 7.94 (d, J=7.9 Hz, 1H), 7.72 (overlapping ddd, 1 H), 7.66 (overlapping ddd, 1H), 7.46 (d, J=8.0 Hz, 1H), 4.04 (q, J=9.3 Hz, 2H), 3.38 (t, J=7.4 Hz, 2H), 1.53 (overlapping qt, 2H), 0.85 (t, J=7.4 Hz, 3H).

### Example 22

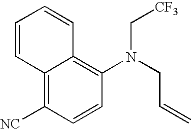

### 4-[2-Propen-1-yl(2,2,2-trifluoroethyl)amino]-1-naphthalenecarbonitrile

Synthesized as described in example 21 from example 19 and allyl bromide: ¹H NMR (400 MHz, MeOH-d₄) δ 8.33 (d, J=8.4 Hz, 1H), 8.16 (d, J=8.4 Hz, 1H), 7.94 (d, J=8.0 Hz, 1H), 7.74 (overlapping ddd, 1H), 7.68 (overlapping ddd, 1H), 7.45 (d, J=8.0 Hz, 1H), 5.88 (ddt, J=17.0, 10.3, 6.5 Hz, 1H), 5.28-5.17 (m, 2H), 4.04 (q, J=9.2 Hz, 2H), 3.96 (d, J=6.5 Hz, 2H).

### Example 23

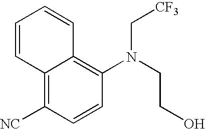

### 4-[(2-Hydroxyethyl)(2,2,2-trifluoroethyl)amino]-1-naphthalenecarbonitrile

A mixture of example 19 (1.00 g, 4.00 mmol), methyl bromoacetate (1.89 mL, 20.0 mmol) and anhydrous K₂CO₃ (2.76 g, 20.0 mmol) in dry MeCN (10 mL) was refluxed under nitrogen for 22 h. The mixture was cooled, Cs₂CO₃ (6.5 g, 20 mmol) and additional methyl bromoacetate (1.89 mL, 20 mmol) were added, and reflux was resumed. After 14 h, the mixture was cooled, poured into water and the whole extracted with EtOAc (×3). The combined organic portions were washed with brine, dried over Na₂SO₄, filtered and concentrated to dryness, affording 0.883 g of a colorless solid that was used without further purification. The crude solid was dissolved in dry THF (10 mL), cooled to 0° C., and a solution of LiBH₄ in THF (2.74 mL of a 2.0 M solution, 5.49 mmol) was added dropwise over 3 min. The cooling bath was removed and the mixture stirred 72 h at room temp. The mixture was cooled to 0° C., quenched by dropwise addition of saturated NH₄Cl, poured into water and the whole extracted with EtOAc (×3). The combined organic portions were washed (water, brine), dried over Na₂SO₄, filtered and concentrated in vacuo. The residue was purified by flash chromatography (EtOAc/hexanes), affording 0.638 g of the title compound as a colorless syrup which slowly solidified: MS (APCI) m/z 295 (M+1).

Compounds of the current invention are modulators of the androgen receptor, glucocorticoid receptor, the mineralocorticoid receptor, and/or the progesterone receptor. Activity mediated through these oxosteroid nuclear receptors was determined using the following in vitro and in vivo assays.

In Vitro Assays:

The following abbreviations and sources of materials are used
Fluormone PL Red—a commercially available PR fluoroprobe (PanVera Corp, Product No P2965)
Fluormone GS Red—a commercially available GR fluoroprobe (PanVera Corp, Product No P2894)
Fluormone AL Green—a commercially available AR fluoroprobe (PanVera Corp, Product No P3010)
PR-LBD—Purified human progesterone ligand binding domain tagged with Glutathione Transferase (PanVera Corp, Product No P2900)
GR—purified human glucocorticoid receptor (PanVera Corp, Product No P2812)
AR-LBD—Purified rat androgen ligand binding domain tagged with Glutathione Transferase (PanVera Corp, Product No P3009)
PR Screening Buffer—100 mM potassium phosphate (pH 7.4), 100 μG/mL bovine gamma globulin, 15% ethylene glycol, 0.02% NaN₃, 10% glycerol (PanVera Corp Product No P2967) with 0.1% wlv CHAPS
AR Screening Buffer—pH 7.5 containing protein stabilizing agents and glycerol (PanVera Corp Product No P3011)
GR Screening Buffer—100 mM potassium phosphate (pH 7.4), 200 mM Na₂MoO₂, 1 mM EDTA, 20% DMSO (PanVera Corp Product No P2814) with GR stabilizing peptide (100 μM) (PanVera Corp Product No P2815)
DTT—dithiothreitol (PanVera Corp Product No P2325)
Discovery Analyst—is an FP reader
DMSO—dimethylsulphoxide
   Progesterone Receptor Fluorescence Polarization Assay:

The progesterone receptor fluorescence polarization assay is used to investigate the interaction of the compounds with the progesterone receptor.

Compounds are added to the 384 well black plates to a final volume of 0.5 μL. Sufficient Fluormone PL Red and PR-LBD are defrosted on ice to give a final concentration of 2 nM and 40 nM, respectively. PR screening buffer is chilled to 4° C. prior to addition of DTT to give a final concentration of 1 mM. The Fluormone PL Red and PR-LBD in PR Screening Buffer are added to compound plates to give a final volume of 10 μL. The assay is allowed to incubate at 20-22° C. for 2 hours. The plates are counted in a Discovery Analyst with suitable 535 nM excitation and 590 nM emission interference filters. Compounds that interact with the PR receptor result in a lower fluorescence polarization reading. Test compounds are dissolved and diluted in DMSO. Compounds are assayed in singlicate, a four parameter curve fit of the following form being applied

$$y = \frac{a - d}{1 + {( \frac{x}{c} )}^{b}} + d$$

where a is the minimum, b is the Hill slope, c is the IC₅₀ and d is the maximum. Maximum and minimum values are compared to adhesion in the absence of compound and in the presence of 10⁻⁵M progesterone. Data is presented as the mean pIC₅₀ with the standard error of the mean of n experiments. Compounds with pIC₅₀ greater than 5.0 and a % max greater than 50 are considered desirable.
Androgen Receptor Fluorescence Polarization Assay:

The androgen receptor fluorescence polarization assay is used to investigate the interaction of the compounds with the androgen receptor.

Compounds are added to the 384 well black plates to a final volume of 0.5 μL. Sufficient Fluormone AL Green and AR-LBD are defrosted on ice to give a final concentration of 1 nM and 25 nM, respectively. AR screening buffer is chilled to 4° C. prior to addition of DTT to give a final concentration of 1 mM. The Fluormone AL Green and AR-LBD in AR Screening Buffer are added to compound plates to give a final volume of 10 μL. The assay is allowed to incubate at 20° C. for 5 hours. The plates are counted in a Discovery Analyst with suitable 485 nM excitation and 535 nM emission interference filters. Compounds that interact with the AR receptor result in a lower fluorescence polarization reading. Test compounds are dissolved and diluted in DMSO. Compounds are assayed in singlicate, a four parameter curve fit of the following form being applied

$$y = \frac{a - d}{1 + {( \frac{x}{c} )}^{b}} + d$$

where a is the minimum, b is the Hill slope, c is the IC₅₀ and d is the maximum. Maximum and minimum values are compared to adhesion in the absence of compound and in the presence of 10⁻⁵M dihydrotestosterone. Data is presented as the mean pIC50 with the standard error of the mean of n experiments. Compounds with pIC₅₀ greater than 5.0 and a % max greater than 50 are considered desirable.
Glucocorticold Receptor Fluorescence Polarization Assay

The glucocorticoid receptor fluorescence polarization assay is used to investigate the interaction of the compounds with the glucocorticoid receptor.

Compounds are added to the 384 well black plates to a final volume of 0.5 μL. Sufficient Fluormone GS Red and GR are defrosted on ice to give a final concentration of 1 nM and 4 nM, respectively. GR screening buffer is chilled to 4 ° C. prior to addition of DTT to give a final concentration of 1 mM. The Fluormone GS Red, and GR in GR Screening Buffer are added to compound plates to give a final volume of 10 μL. The assay is allowed to incubate at 4° C. for 12 hours. The plates are counted in a Discovery Analyst with suitable 535 nM excitation and 590 nM emission interference filters. Compounds that interact with the GR receptor result in a lower fluorescence polarization reading. Test compounds are dissolved and diluted in DMSO. Compounds are assayed in singlicate, a four parameter curve fit of the following form being applied

$$y = \frac{a - d}{1 + {( \frac{x}{c} )}^{b}} + d$$

where a is the minimum, b is the Hill slope, c is the EC₅₀ and d is the maximum. Maximum and minimum values are compared to adhesion in the absence of compound and in the presence of 10⁻⁵ M dexamethasone. Data is presented as the mean pIC₅₀ with the standard error of the mean of n experiments. Compounds with pIC₅₀ greater than 5.0 and a % max greater than 50 are considered desirable.
Transient Transfection Assay:

Cotransfection assays using full-length hAR were performed in CV-1 cells (monkey kidney fibroblasts). The cells were seeded in charcoal-stripped medium in 96-well plates (24,000 cells/well) and incubated overnight. Transient transfections were carried out using the following plasmids: pSG5-AR, MMTV LUC reporter, β-actin SPAP, and pBluescript (filler DNA). The cell plates were then incubated for 6-20 hours. The transfection mixture was washed away and then the cells were drugged with doses ranging from 10⁻¹⁰ to 10⁻⁵. Two replicates were used for each sample. Incubation with drug was continued for 14 hours. A spectrophotometer was used for SPAP measurements, while a topcounter was used to read the results from the luciferase assay. The ratio of luciferase activity to SPAP activity was calculated to normalize the variance in cell number and transfection efficiency.

Data Analysis:

Data were reduced using RoboFit99. The results were expressed as percent of maximum as calculated by the following formulas:

$$fold activation = \frac{( ( \frac{( Luc )}{( SPAP - SPAP substrate blank avg . )} ) - basal activation )}{basal {activation}^{*}}$$

*basal activation per plate=(Luc vehicle)/(SPAP vehicle−substrate blank average)
% max.=(^{fold activation of unknown}/posive control fold activation avg.)×100

Curves were fit from these data using RoboFit to determine EC₅₀'s for agonists and IC₅₀'s for antagonists using the following equation:
Y=((Vmax*x)/(K+x))+Y2

These values were converted to pEC₅₀'s and pIC₅₀'s for posting by using the following equations:
pEC₅₀=−log(EC₅₀)
pIC₅₀=−log(IC₅₀)

For antagonist assays, the percent maximum response antagonist was calculated by the following formula in which Yₘᵢₙ and Yₘₐₓ are curve asymptotes at the maximum or minimum concentration tested:
% max. resp. ant.=100*(1−Yₘᵢₙ/Yₘₐₓ)

For antagonist assays, pKb's were calculated using the following formula:
pKb=IC₅₀ of unknown/((1+*conc.*)/DHT EC₅₀ average)
where *conc.*=concentration of DHT used as the agonist in the medium for the antagonist experiment, expressed in nM. This concentration was set at twice pEC₅₀. This would be 0.2 for AR.

Compounds with a pXC₅₀ greater than 5.0 are considered desirable.

Castrated Male Rat Model (ORX Rat)

The activity of the compounds of the present invention as modulators of the androgen receptor was investigated using a castrated male rat model (ORX) as described in C. D. Kockakian, Pharmac. Therap. B 1(2), 149-177 (1975); C. Tobin and Y. Joubert, Developmental Biology 146, 131-138 (1991); J. Antonio, J. D. Wilson and F. W. George, J Appl. Physiol. 87(6) 2016-2019 (1999)) the disclosures of which herein are included by reference.

It has been well defined that androgens play important roles in the maintenance and growth of many tissues in both animals and humans. Muscles, like the levator ani and bulbocavernosus, and sexual accessory organs, such as the prostate glands and seminal vesicles have high expression levels of the androgen receptor and are known to respond quickly to exogenous androgen addition or androgen deprivation through testicular ablation. Castration produces dramatic atrophy of muscle and sexual accessory organs; whereas the administration of exogenous androgens to the castrated animal results in effective hypertrophy of these muscles and sexual accessory organs. Although the levator ani muscle, also known as the dorsal bulbocavernosus, is not ‘true skeletal muscle’ and definitely sex-linked, it is reasonable to use this muscle to screen muscle anabolic activities of test compounds because of its androgen responsiveness and simplicity of removal.

Male Sprague-Dawley rats weighing 160-180 grams were used in the assay. The rats were singly caged upon receiving and throughout the study. Bilateral orchidectomies were performed in sterilized surgical conditions under isoflurane anesthesia. An anteroposterior incision was made in the scrotum. The testicles were exteriorized and the spermatic artery and vas deferens were ligated with 4.0 silk 0.5 cm proximal to the ligation site. The testicles then were removed by a surgical scissors distal to the ligation sites. The tissue stumps were returned to the scrotum, the scrotum and overlying skin were closed by a surgical stapler. The Sham-ORX rats underwent all procedures except ligation and scissors cutting. The rats were assigned randomly into study groups 7-10 days post surgery based on the body weight.

Dihydrotestosterone (DHT) was used as a positive control (1-10 mg/kg s.c.). Compounds of the current invention were administered subcutaneously or orally for 4-28 days. The rats were weighed daily and doses were adjusted accordingly. The general well being of the animal was monitored throughout the course of the study.

At the end of the study, the rats were euthanized in a CO₂ chamber. The ventral prostate glands(VP), seminal vesicles(SV), levator ani muscle(LA) and lo bulbocavernosus(BC) were carefully dissected. The tissues were blotted dry, the weights were recorded, and then saved for histological and molecular analysis. The VP and SV weights serve as androgenic indicators and LA and BC are anabolic indicators. The ratio of anabolic to androgenic activities was used to evaluate the test compounds. Serum luteinizing hormone(LH), follicle stimulating hormone(FSH) and other potential serum markers of anabolic activities were also analyzed.

In general, desirable compounds show levator ani hypertrophy and very little prostate stimulation

Test compounds were employed in free or salt form.

All research complied with the principles of laboratory animal care (NIH publication No. 85-23, revised 1985) and GlaxoSmithKline policy on animal use.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included within the scope of the appended claims.

What is claimed is:

1-7. (canceled)

8. A compound of Formula (I) 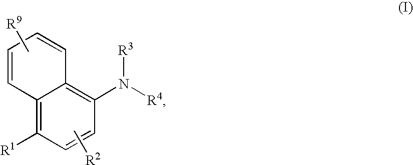
or a salt or solvate thereof, wherein
R¹ is cyano, nitro, halogen, haloalkyl, heterocyclyl, hydroxy, alkoxy, haloalkoxy, —OC(O)⁶, —CO₂R⁶, —CONHR⁶, —C(O)R⁶, —S(O)ₙR⁶, —SO₂N(R⁶)₂, —NHC(O)R⁶, or —NHSO₂R⁶;
R² is H, cyano, nitro, halogen, haloalkyl, alkyl, alkenyl, alkynyl, hydroxy, alkoxy, haloalkoxy, —OC(O)R⁶, or aryl;
R³ and R⁴ each are independently —(CH₂)ₓ—R⁵,
where x is 0 to 6, and
R⁵ is selected from H, alkyl, hydroxy, haloalkyl, alkenyl, alkynyl, cycloalkyl, heterocyclyl, —C(O)OR⁷, or —N(R⁸)₂;
each R⁶ independently is H, alkyl, alkenyl, alkynyl, cycloalkyl, heterocyclyl, aryl, aralkyl, heteroaryl, or heteroaralkyl;
each R⁷ independently is H, alkyl, cycloalkyl, aryl;
each R⁸ independently is H or alkyl; and R⁹ is H, cyano, nitro, halogen, haloalkyl, alkyl, alkenyl, alkynyl, hydroxy, alkoxy, haloalkoxy, —OC(O)R⁸, or aryl;
and the compound is selected from:
N-(cyclopropylmethyl)-N-(4-nitro-1-naphthyl)-N-propylamine;
N-cyclohexyl-N-methyl-4-nitro-1-naphthalenamine;
N-(4-nitro-1-naphthyl)-N,N-dipropylamine;
N-butyl-N-methyl-N-(4-nitro-1-naphthyl)amine;
4-[ethyl(2-methyl-2-propenyl)amino]-1-naphthonitrile;
N-butyl-N-ethyl-4-nitro-1-naphthalenamine;
4-[butyl(methyl)amino]-1-naphthonitrile;
4-[(cyclopropylmethyl)(propyl)amino]-1-naphthonitrile;
N¹-ethyl-N²,N²-dimethyl-N′-(4-nitro-1-naphthyl)-1,2-ethanediamine;
4-(propylamino)-1-naphthonitrile;
4-[(3-hydroxypropyl)amino]-1-naphthonitrile;
3-[(4-nitro-1-naphthyl)amino]propan-1-ol;
4-[(cyclopropylmethyl)amino]-1-naphthalenecarbonitrile;
4-{(cyclopropylmethyl)[3-(1-piperidinyl)propyl]amino}-1-naphthalene carbonitrile trifluoroacetate;
4-[(cyclopropylmethyl)(3-hydroxypropyl)amino]-1-naphthalenecarbonitrile;
4-nitro-N-(2,2,2-trifluoroethyl)-1-naphthalenamine;
4-bromo-N-(2,2,2-trifluoroethyl)-1-naphthalenamine;
4-bromo-N,N-bis(2,2,2-trifluoroethyl)-1-naphthalenamine;
4-[(2,2,2-trifluoroethyl)amino]-1-naphthalenecarbonitrile;
4-[bis(2,2,2-trifluoroethyl)amino]-1-naphthalenecarbonitrile;
4-[propyl(2,2,2-trifluoroethyl)amino]-1-naphthalenecarbonitrile;
4-[2-propen-1-yl(2,2,2-trifluoroethyl)amino]-1-naphthalenecarbonitrile; and
4-[(2-hydroxyethyl)(2,2,2-trifluoroethyl)amino]-1-naphthalenecarbonitrile.

9. (canceled)

10. A pharmaceutical composition comprising a compound according to claim 8, and a pharmaceutically acceptable carrier.

11-15. (canceled)

16. A method for the treatment or prophylaxis of conditions or disorders that respond to selective androgen receptor modulation comprising the administration of a compound according to claim 8.

17. A method for the treatment or prophylaxis of osteoporosis, muscle wasting, frailty, cardiovascular disease, breast cancer, uterine cancer, prostatic hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM comprising the administration of a compound according to claim 8.
